(19) United States
(12) Patent Application Publication
(10) Pub. No.: US 2007/0202595 A1
(43) Pub. Date: Aug. 30, 2007

(54) Human embryonic stem cell lines and methods of use thereof

(76) Inventors: Carlos Simon Valles, Godella (Valencia) (ES); Antonio Pellicer Martinez, Valencia (ES); Ruben Moreno Palanques, Valencia (ES)

Correspondence Address: WOLF GREENFIELD & SACKS, P.C., 600 ATLANTIC AVENUE, BOSTON, MA, 02210-2206, US

(21) Appl. No.: 11/655,048

(22) Filed: Jan. 18, 2007

Related U.S. Application Data
(63) Continuation of application No. PCT/ES06/00017, filed on Jan. 18, 2006.
(60) Provisional application No. 60/759,655, filed on Jan. 18, 2006.

(51) Int. Cl. C12N 5/08

(52) U.S. Cl. 435/366

(57) ABSTRACT

The invention provides novel human embryonic stem cell lines. The invention further provides human embryonic stem cell lines that are genetically related and immunologically identical. The invention further provides methods of deriving, propagating and using such lines, optionally under animal-free conditions.

### RELATED APPLICATIONS

This application is a continuation of International Patent Application Serial No. PCT/ES2006/000017, filed Jan. 18, 2006. This application also claims priority to U.S. provisional patent application Ser. No. 60/759655, filed Jan. 18, 2006. Both applications are incorporated herein by reference.

### FIELD OF THE INVENTION

The invention relates to human embryonic stem cell lines and methods of use thereof.

### BACKGROUND OF THE INVENTION

Human embryonic stem cells (hESCs) have the ability to differentiate into various cell lineages in vitro and in vivo including but not limited to melanocytes, hematopoietic cells, hepatocytes, kidney cells, skeletal muscle cells, dopaminergic neurons, glial cells, cardiomyocytes, endothelial cells, and osteoblasts. hESCs cells are therapeutically attractive because of this pluripotency. The use of such cells and their differentiated progeny is contemplated for the treatment of various conditions. However, there are some current drawbacks to the currently existing hESC lines.

First, transplant of hESCs (and their differentiated progeny) into human subjects will require, in most instances, histocompatibility between the hESCs and the human recipients of such cells. Given the level of diversity in human histocompatibility antigens, it has been estimated that 150,000 lines would need to be generated.

Second, therapy of human subjects with hESC lines (and their differentiated progeny), including in a transplant setting, will also. almost certainly require that the lines be generated in the absence of animal (i.e., non-human) products. The first report of hESC used mouse fibroblast feeder cells to derive and culture the cells. See U.S. Pat. Nos. 5,843,780 and 6,200,806. Such an approach can result in the transmission of mouse pathogens into human subjects.

Third, although there are reportedly on the order of 75-100 hESC lines currently in existence, only a few of these have been confirmed to have true stem cell properties including self renewal (as evidenced by long term culture) and pluripotent lineage differentiation.

There exists a need for more hESC lines in order to overcome these issues.

### SUMMARY OF THE INVENTION

The invention is premised, in part, on the derivation and characterization of hESC lines from long term cryopreserved human embryos. The lines were derived in defined medium without serum and without animal feeder cells (i.e., animal-free conditions). Two examples of these lines are designated VAL-1 and VAL-2. These lines were derived from “sister” embryos (i.e., embryos having identical “parentage”) and are immunologically identical. Accordingly, these lines can be used together, for example in a therapeutic setting, due to their histocompatibility.

Thus, in one aspect, the invention provides an isolated human embryonic stem cell population wherein said cell population (1) has a normal karyotype after at least 85 passages; (2) expresses Stage Specific Embryonic Antigen (SSEA) 4, Tumor Rejection Antigen (TRA)-1-60 and TRA-1-81; (3) is alkaline phosphatase positive; (4) expresses Oct-4, Rex-1, Cripto, Thy-1 and Nanog; (5) is negative for Matn1, Amylase and Dbh; and (6) has telomerase activity. These characteristics (or properties) are preferably maintained over the long term (e.g., after the at least 85 passages).

In other aspects, the invention provides an isolated human embryonic stem cell population wherein said cell population has the characteristics of embryonic stem cell line VAL-1 or VAL-2.

In still other aspects, the invention provides an isolated human embryonic stem cell population wherein said cell population is embryonic stem cell line VAL-1 or VAL-2, and progeny thereof. The progeny may be undifferentiated or differentiated progeny including but not limited to cardiac, neural, muscular, and hematopoietic differentiated progeny.

In some embodiments, the population comprises genetically modified cells.

In some embodiments, the population is passaged at least 50 times, at least 75 times, at least 100 times, or more. In one embodiment, the population is passaged at least 85 times or at least 120 times.

In some embodiments, the population has been cryopreserved, and optionally thawed and passaged, with no change in marker expression, karyotype or telomerase activity. The population may be cryopreserved for more than 1 year.

The human embryonic stem cell “populations” referred to herein are also referred to as human embryonic stem cell “lines”.

In another aspect, the invention provides a pair of isolated human embryonic stem cell populations (or lines) that express HLA markers A2, A23, B44, B40, CW4, CW5, DR7, DR15, DQ2 and DQ6. The invention provides compositions and methods for using the pair of human embryonic stem cell populations (and/or their differentiated progeny). In one aspect, the pair of lines (and/or their differentiated progeny) are used in human subjects having one or more of the afore-mentioned HLA markers. The populations (and/or their differentiated progeny) may be introduced into the subjects at the same or at different times. The methods may further comprise use of different differentiated populations from the lines in the same subject. As an example, a subject may receive VAL-1 derived hematopoietic cells and VAL-2 derived keratinocytes at the same or at different times.

In still another aspect, the invention provides compositions comprising one or more human embryonic stem cells from any of the foregoing human embryonic stem cell populations. The composition may be a pharmaceutical preparation, but it is not so limited.

In still another aspect, the invention provides a culture comprising any of the foregoing stem cell populations. The culture may comprise feeder cells, preferably human feeder cells such as but not limited to human placental feeder cells. In certain embodiments, the feeder cells are mitotically inactivated, such as for example by irradiation. The culture may be a serum-free culture. The culture may comprise fibroblast growth factor (FGF) such as but not limited to basic FGF (bFGF). In some embodiments, the culture may be feeder-free. In preferred embodiments, the culture is free of animal (i.e., non-human) products. The culture may comprise human embryonic stem cells and differentiated progeny thereof.

In yet another aspect, the invention provides a method of culturing including propagating a human embryonic stem cell populations comprising culturing any of the foregoing human embryonic stem cell populations in a serum-free medium and optionally in the presence of human feeder cells. The human feeder cells may be human placental feeder cells. The feeder cells may be mitotically inactivated for example by irradiation. The medium may comprise FGF such as but not limited to bFGF. In some embodiments, bFGF is present in an amount of about 1-15 ng/mL or 1-10 ng/mL.

In still another aspect, the invention provides a method of differentiating a human embyronic stem cell population in vitro comprising exposing any of the foregoing human embryonic stem cell populations to differentiation conditions for a time sufficient to allow differentiation of the human embryonic stem cell population into differentiated cells. Depending on the embodiment, the differentiation conditions may comprise feeder cells or may be feeder-free. In some embodiments, the differentiation conditions comprise one or more factors selected from the group consisting of retinoic acid, epidermal growth factor (EGF), bone morphogenic protein 4 (BMP4), fibroblast growth factor (FGF), steroid hormones, activin-A, transforming growth factor-beta 1 (TGF-β1), hepatocyte growth factor (HGF), and nerve growth factor (NGF). The method may further comprise introducing the differentiated cells into a subject such as but not limited to a human subject.

In still another aspect, the invention provides a method of differentiating a human embryonic stem cell population comprising introducing any of the foregoing human embryonic stem cell populations into a subject, including by not limited to a human subject. In some embodiments, the subject has a condition affecting the liver, muscle, skin, brain, nervous system, heart, circulatory system, hematopoietic system, pancreas or bone. In some embodiments, the population is introduced into the subject by local administration, such as but not limited to administration to an organ or a tissue. In some embodiments, the population is introduced into the subject by systemic administration, such as but not limited to intravenous administration. In some embodiments, the method further comprises exposing the population to differentiation conditions in vitro prior to introduction in the subject. In some embodiments, the subject expresses one, two, three, four, five, six, seven, eight or nine HLA markers selected from the group consisting of A2, A23, B44, B40, CW4, CW5, DR7, CD15, DQ2 and DQ6. In some embodiments, the subject expresses all of the foregoing markers. In some embodiments, the subject receives VAL-1 and VAL-2 cells or progeny thereof.

In yet another aspect, the invention provides a method for determining therapeutic efficacy or cytotoxicity of a compound comprising exposing any of the foregoing human embryonic stem cell lines to a compound (e.g., contacting the line to the compound), and determining an effect of the compound on the human embryonic stem cell line. The line may be differentiated prior to contact with the compound. The differentiation may be hematopoietic differentiation or neural differentiation, but the invention is not so limited. The effect of the compound may be cell death, cell growth (e.g., increase in cell number), increase in the number of undifferentiated human embryonic stem cells, decrease in the number of undifferentiated human embryonic stem cells (e.g., due to differentiation), increase in the number of cells of a particular differentiated lineage, changes in expression profiles, and the like.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE FIGURES

FIGS. 1A-F are a series of photographs showing the morphologic features of VAL-1 and VAL-2 derivation. Two different embryos (A, D) were thawed and adhered on human placental fibroblasts. After 18 (B) and 21 (E) days of derivation, the cell colonies had the typical hESC appearance, which was maintained after at least 90 days of cell culture (C, F).

FIGS. 2A-J show various characteristics of VAL-1 and VAL-2 including karyotypes (A, F), immunostaining for Stage-Specific Embryonic Antigen 4 (B, G), TRA-1-60 (C, H) and TRA-1-81 (D, I), and phosphatase alkaline assay (E, J). Both cell lines had normal karyotypes and were positive for all non-differentiation (i.e., immature) markers tested.

FIG. 3 shows the telomerase activity of VAL-1 and VAL-2 hESC lines. The negative control was obtained by heat inactivation in both lines. The 36 bp band corresponds to the internal polymerase chain reaction control, and the 50 bp band corresponds to telomerase activity, which increases in 6 bp bands in immortal cells.

FIG. 4 is a GeneScan™ printout demonstrating genetic fingerprinting profiles of VAL-1 (third row), VAL-2 (fourth row), maternal (egg donor) (first row) and paternal (sperm donor) (second row) donor samples.

FIG. 5 is a GeneScan™ printout demonstrating genetic fingerprinting profiles of VAL-1 and VAL-2. The Figure shows a detailed analysis of loci D35 1358 and VWA from the third and fourth rows of FIG. 4.

FIG. 6 is a genomic profile comparison of VAL-1 and VAL-2 demonstrating that the lines are not genetically identical to each other. The circled data points represent variant or disparate genomic loci levels between the lines.

FIGS. 7A-D are photographs of differentiated myocardiocytes spontaneously derived from VAL-1.

FIGS. 8A-C are photographs of immunostained spontaneously differentiated progeny from VAL-1 and VAL-2. FIG. 8B shows B-tubulin expression in neuron-like cells. FIG. 8C shows actin expression in muscle-like cells.

FIGS. 9A-D are photographs of teratomas formed after intratesticular injection of VAL-1 and VAL-2 into SCID mice.

FIGS. 10A-L are photographs showing histology from a VAL-1 derived teratoma.

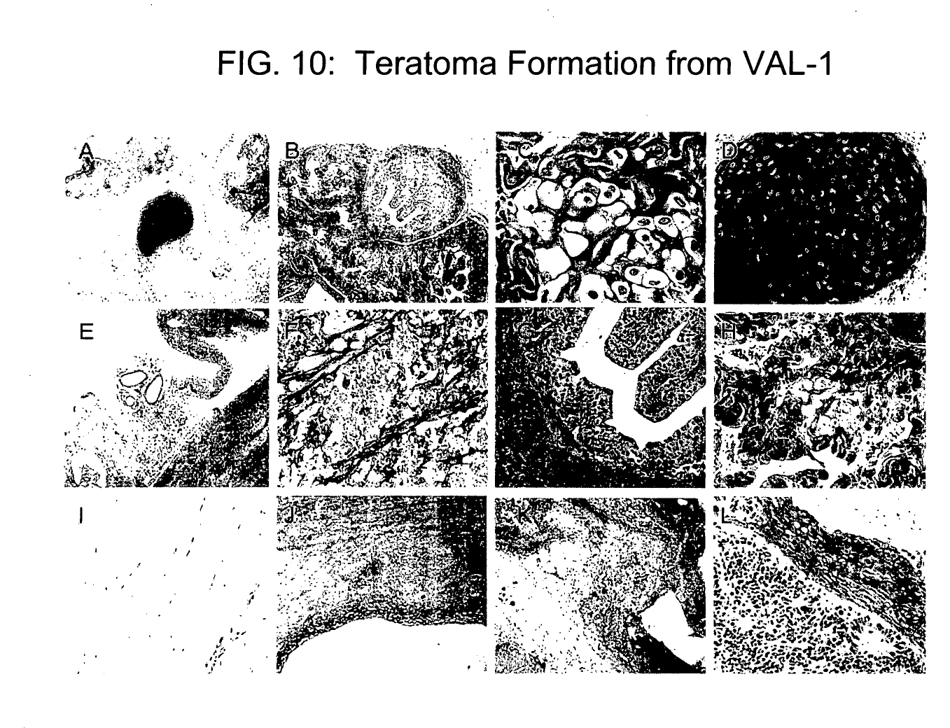

It is to be understood that the drawings are not required for enablement of the invention.

### DETAILED DESCRIPTION OF THE INVENTION

The invention is premised, in part, on the derivation, characterization and use of ESC lines from human embryos. These lines were derived from human embryos that had been cryopreserved for several years. The invention provides a molecular and functional characterization of these lines and thereby embraces embryonic stem cell lines having similar characteristics. The invention further embraces methods of manipulating such lines including culturing (including propagating), long term storage (including cryopreservation), testing (e.g., in a screening assay), and differentiation of such lines.

The hESC lines were generated from embryos with identical parentage (i.e., made from the same progenitors (i.e., egg and sperm sources)). Accordingly, the embryos and the lines are referred to herein as “sister” embryos and lines. In addition, however, the lines are also immunologically identical to each other, as described herein. As a result, the invention envisions the combined use of such lines in treatment regimens given their histocompatibility.

The invention provides ESC lines of human origin. As used herein, an ESC line is a cell line derived in vitro from an embryo and having stem cell-like properties. The stem cell properties of the hESC lines of the invention include one or more of the genotypic, phenotypic and functional properties disclosed herein. Preferably, the line maintains such properties in the long term and through various passages, cultures, and freezing and thawing cycles. For example, VAL-1 has been passaged at least 120 times and VAL-2 has been passaged at least 85 times with maintenance of their phenotypic and functional properties.

Genotypic properties can be gross such as karyotype, or more detailed such as genomic profiles. The lines provided herein preferably contain a normal karyotype. Specific embodiments of these lines have a 46, XX karyotype. Karyotype analysis is known in the art and is routine for ordinarily skilled artisans. Phenotypic properties include morphological properties such as a small round cell shape with high nucleus to cytoplasm ratio and prominent nucleoli. Phenotypic properties also include intracellular or cell surface marker expression. These properties include expression of immature or non-differentiation markers such as Stage-Specific Embryonic Antigen 4 (SSEA 4), the keratin sulphate-associated antigens Tumor Rejection Antigen (TRA)-1-60 and TRA-1-81, stem cell transcription factor Oct-4, Rex-1, Cripto, Thy-1 and/or Nanog. hESC lines can also be characterized according to non-expression of certain markers such as differentiation markers. To this end, the lines provided herein are characterized as not expressing the differentiation markers Matn1, Amylase and Dbh. Other phenotypic properties include the presence of alkaline phosphatase and telomerase.

Phenotypic markers can be assayed in a number of ways. For example, they may be assayed using marker-specific binding agents such as marker-specific antibodies or fragments thereof. For example, SSEA-4 can be detected by immunostaining using monoclonal antibodies such as MC-813-70 (Salter and Knowles, 1979) which is commercially available from Chemicon (Temecula, Calif.). TRA-1-60 and TRA-1-81 expression can be detected using monoclonal antibodies available from commercial sources such as Chemicon (Temecula, Calif.) also. Nanog can also be detected using antibodies such as AF1997 available from R&D Systems (Minneapolis, Minn.). Oct-4 can also be detected using antibodies such as AF1759 from R&D Systems (Minneapolis, Minn.), or sc-8628 or sc-9081 available from Santa Cruz Biotechnology, Inc. (Santa Cruz, Calif.).

Phenotypic markers can also be assayed using reverse transcriptase polymerase chain reaction (RT-PCR). Markers such as Oct-4, Rex-1, Cripto, Thy-1, Nanog, Matn1, Amylase, and Dbh can be detected using RT-PCR. Primers for use in RT-PCR amplification of these various markers are known in the art. (See for example Brimble et al. Stem Cells and Development, 13:585-596, 2004; Noaksson et al. Stem Cells, 23:1460-1467, 2005.) Alternatively, such primers can be designed by one of ordinary skill in the art using routine experimentation. For example, as shown in the Examples, primers can be designed using freely available web-based software (Primer3, Genefisher).

Still other phenotypic markers can be detected using enzymatic assays. Such markers include alkaline phosphatase and telomerase. Alkaline phosphatase may be detected in a standard assay using reagents available from commercial sources such as Chemicon (Temecula, Calif.) or Vector Laboratories (Burlingame, Calif.; Vector Blue/Red Alkaline Phosphatase Substrate Kit). Telomerase activity can be detected using commercially available telomerase detection kits such as those available from Chemicon (TRAPEZE Telomerase Detection Kit, Temecula, Calif.), optionally together with DNA gel staining such as but not limited to SYBR gel stain (Molecular Probes, Eugene, Ore.).

Functional properties of hESCs include the ability to form compact colonies in vitro, the ability to be cultured long term in vitro, and importantly, the ability to maintain stem cell characteristics in the long term such as but not limited to pluripotency, as described herein. These properties can be assayed visually, or by culture and/or differentiation in vitro or in vivo.

hESCs are at least pluripotent and in some cases may be totipotent. Pluripotency refers to the ability of these cells to generate most if not all tissues in an organism. Totipotency refers to the ability of these cells to generate an entire organism. The hESCs of the invention are able to differentiate into mesoderm, endoderm and ectoderm lineages. Accordingly, hESCs can differentiate into at least one mesoderm lineage such as bone, cartilage, smooth muscle, cardiac muscle, skeletal muscle, kidney, striated muscle and hematopoietic cells; at least one endoderm lineage such as liver, pancreas, thyroid, primitive gut and respiratory epithelium; and at least one ectoderm lineage such as skin, pigment cells such as melanocytes, neurons, glial cells, hair follicles and tooth buds. hESC may also differentiate into germ cells.

The hESC lines can exist in culture for extended periods of time (e.g., up to a year or more, and potentially indefinitely) without differentiating completely and without exhaustion, and with maintenance of their original phenotype, for example as described herein.

Accordingly, the invention provides hESC lines having one or more of the foregoing characteristics. Preferably, the hESC lines possess all of the foregoing characteristics, however the invention is not so limited.

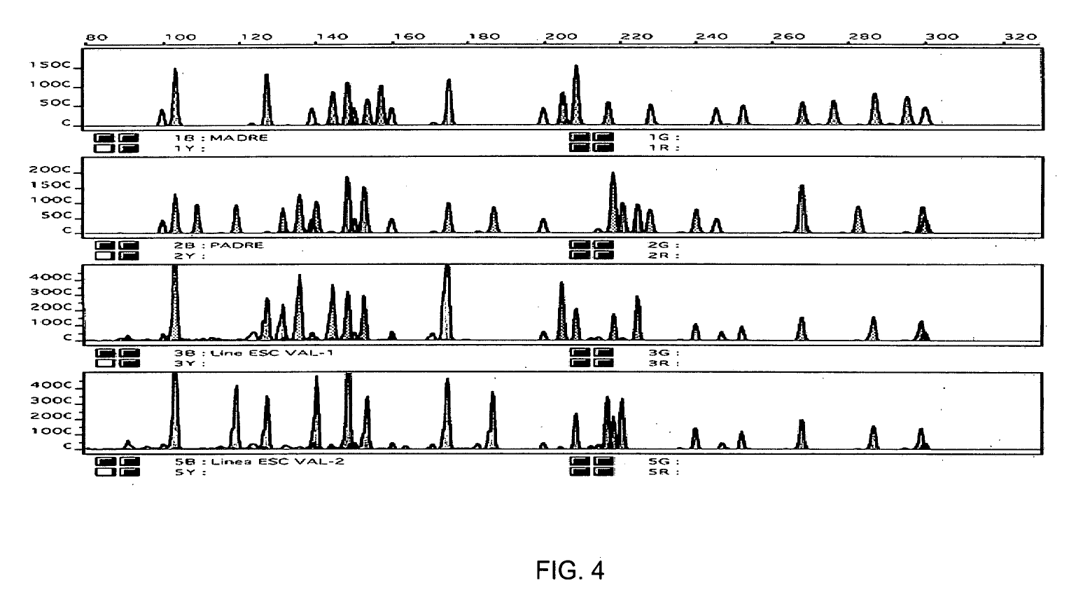

The hESC lines provided by the invention include those designated VAL-1 and VAL-2. VAL-1 and VAL-2 are sister cell lines in the sense that they derive from sister embryos (i.e., embryos having the same “parents”). Genetic marker fingerprinting profiles for the parents and cell lines are shown in FIG. 4. Nine different polymorphic markers which have independent hereditary transmission (i.e., D3S1358, vWA, FGA, D8D1179, D21S11, D18S51, D5S818, D13S317, D7S820) were analyzed. The data demonstrate with 99% likelihood that VAL-1 and VAL-2 cell lines derived from source of the maternal and paternal samples shown, and correspondingly that VAL-1 and VAL-2 are sister cell lines.

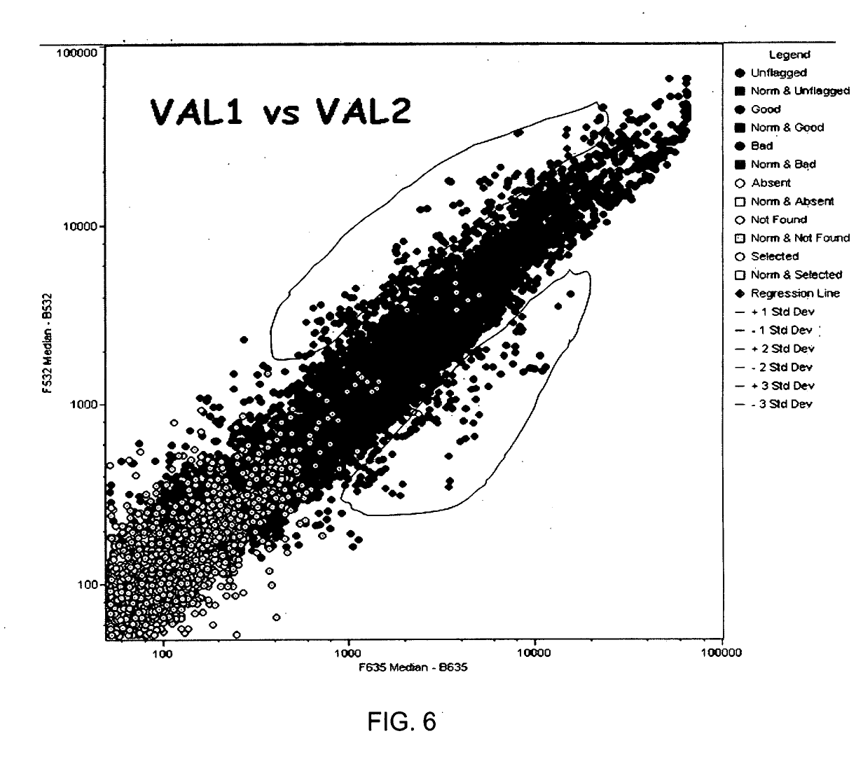

VAL-1 and VAL-2 are also immunologically identical. The lines share identical HLA markers A2, A23, B40, B44, CW4, CW5, DR7, DR15, DQ2 and DQ6. The lines are however genetically different as demonstrated by the genomic profile comparison of FIG. 6. The cells therefore can be used to determine the relevance of such genetic differences on the functionality of the lines such as but not limited to differentiation capacity.

In some instances, the invention provides the hESC lines in an isolated form. As used herein, isolated means that the cells are physically separated from their naturally occurring environment such as a blastocyst, an ICM, and components thereof. The lines are generally provided as a clonal population.

The hESC lines of the invention may be further characterized via the method of their derivation. VAL-1 and VAL-2 were derived from long term (i.e., 5 years or more) cryopreserved embryos at the blastocyst stage. Day 2 human embryos cryopreserved and donated for research were thawed and then treated to remove the zona pellucida using for example acid Tyrode's solution. The embryos were washed in medium (e.g., 80% DMEM, 20% serum-free knockout serum replacement (GibcoBRL), optionally supplemented with 0.1 mM nonessential amino acids, 0.1 mM beta-mercaptoethanol, and 1 mM L-glutamine), optionally containing 12 ng/mL human basic fibroblast growth factor (Invitrogen). The embryos were then transferred onto human feeder cells. Suitable human feeder cells include but are not limited to human placental fibroblasts. These feeder cells were mitotically inactivated. Mitotic activation can be accomplished using for example irradiation or chemicals (e.g., mitomycin C). The cultures were maintained for 2-3 weeks, at which time the initial colonies were mechanically disrupted and allowed to re-attach to the feeder cells. After about one week, the colonies were again mechanically disrupted and then transferred to fresh feeder cells. hESCs were identified morphologically as round cells with prominent nucleoli. Individual colonies were isolated and recultured in order to achieve a clonal population, after which the lines were capable of at least 50, 75, 100, or more passages.

The invention contemplates derivation of the hESC lines provided herein in other ways. As an example, the hESC lines may be generated from freshly prepared embryos or embryos that have been cryopreserved for the short term only (e.g., days, weeks or months). As another example, the derivation process may involve isolation of ICM cells from a blastocyst without treatment with acid Tyrode's solution.

The invention contemplates compositions comprising the hESCs provided herein. Such compositions may comprise other components such as human feeder cells (e.g., human placental feeder cells), progeny of the stem cell lines including differentiated progeny, differentiation factors, extracellular matrices, pharmaceutically acceptable carriers, and the like. These compositions may include cultures of the hESCs. Such cultures may include human or animal serum, or they may be serum-free. For example, the cultures may comprise serum replacements, as disclosed herein.

The invention therefore further contemplates methods of culturing including propagating undifferentiated hESC lines, optionally for weeks, months or years. As discussed in the Examples, these culture methods and conditions are similar to the derivation methods and conditions provided herein. The lines may be cultured in the presence of feeder cells, preferably human feeder cells, and even more preferably human placental feeder cells as described by Genbacev et al. Fertil Steril. 2005, 83:1517-29. Alternatively, the cells may be cultured in the presence of other types of human feeder cells including but not limited to cells from fetal and adult muscle, skin, fallopian tube epithelium, glandular endometrium, stromal endometrium, marrow stromal and foreskin. Suitable culture medium may comprise DMEM with 20% knockout serum replacement, and optional supplementation with nonessential amino acids, beta-mercaptoethanol, and L-glutamine. Other base media include G2.2, S2 (Scandanavian-2), and the like. In still other embodiments, the disclosed lines may be cultured under feeder-free conditions, optionally in the presence of one or more growth factors that substitute for the feeder cells. Such factors include fibroblast growth factor (FGF) in any one of its various forms or homologs thereof including acid FGF (or aFGF or FGF1) and basic FGF (or bFGF or FGF2). In some important embodiments, the FGF is bFGF. The amount of FGF may vary and one of ordinary skill in the art has the ability to determine the amount required for derivation and/or culture in an undifferentiated state. Suitable ranges include 1-1000 ng/mL, 1-100 ng/mL, 1-15 ng/mL, 1-10 ng/mL, and 1-5 ng/mL. Human FGF is preferred in some embodiments. The hESCs can be propagated in culture indefinitely with regular passaging, optionally onto fresh feeder cells.

The derivation and propagation methods provided herein in some instances use feeder cells, such as human feeder cells, and do not require the use of animal serum. The probability of cross-species contamination using these methods is therefore low to non-existent. Accordingly, the hESC lines and compositions thereof can be further characterized by the absence of animal pathogens and animal cells or cell byproducts.

The invention provides for use of the generated hESC lines within months or years of their derivation. The hESC lines therefore may be stored indefinitely such as by cryopreservation. Methods for cryopreserving ESC are known in the art. Freezing of the cells can be carried out using methods including but not limited to conventional slow freezing methods using dimethyl sulfoxide (DMSO, preferably 10%) as a cryoprotectant (as described by Bongso et al. Hum. Reprod. 9 (11):2110-2117, 1994), vitrification methods (as described by Reubinoff et al. Hum. Reprod. 16 (10):2187-2194, 2001), as well as other methods such as those described by Ji et al. Biotechnol Bioeng (2004), 5:299-312, and Richards et al. Stem Cells (2004);22:779-789. It is to be understood however that in some embodiments the hESC lines may be used prior to cryopreservation, and directly from culture. The invention is not limited in this manner.

The hESC lines can be used for both research and therapeutic purposes. The lines may be differentiated into one or more lineages. The hESCs themselves and/or their progeny may be used therapeutically. Alternatively, the hESCs and/or their progeny may be used in vitro for a number of purposes including screening and identification of self-renewal factors and differentiation factors, and testing of various other factors including putative therapeutic candidate compounds.

The invention contemplates methods of differentiating the hESC lines into one or more particular lineages including but not limited to endothelial cells, neurons, hematopoietic cells, cardiomyocytes, skeletal muscles, hepatocytes, insulin-producing cells, glial progenitor cells, osteoblasts, gametes and kidney cells. The hESC lines may be used to regenerate a specific cell lineage(s), tissue or organ. The invention further embraces the resultant differentiated progeny which include bone, cartilage, smooth muscle, cardiac muscle, skeletal muscle, kidney, striated muscle and hematopoietic cells (mesodermal lineages), liver, pancreas, thyroid, primitive gut and respiratory epithelium (endodermal lineages), skin, pigment cells such as melanocytes, neurons, glial cells, hair follicles and tooth buds (ectodermal lineages), and uses thereof.

Differentiation may occur in vitro or in vivo depending on the application. Methods for differentiating hESCs in vitro have been described. hESCs are differentiated by exposure to differentiation conditions for a time sufficient to produce differentiated cells. As used herein, differentiation conditions are conditions that induce hESCs to differentiate into one or more lineages. These conditions may vary according to the lineage desired. Generally, these conditions may embrace the absence of feeder cells used to maintain the hESCs in an undifferentiated form, changes in cell seeding density, and/or introduction of one or more growth factors and/or other feeder cells that stimulate differentiation into particular lineages. Growth factors that may induce differentiation of hESCs into particular lineages include but are not limited to retinoic acid, epidermal growth factor (EGF), bone morphogenic protein 4 (BMP4), fibroblast growth factor (FGF), steroid hormones (e.g., glucocorticoids, vitamin A, thyroid hormone, androgens, estrogens, and the like), activin-A (mesoderm), transforming growth factor-beta 1 (TGF-β1) (mesoderm), hepatocyte growth factor (HGF), and nerve growth factor (NGF).

Differentiation into cardiac muscle may be induced using retinoic acid, 5-azacytidine, and ascorbic acid. Differentiation into hematopoietic lineages may be induced using bone marrow stromal cells as described in U.S. Pat. No. 6,613,568 and/or early-acting hematopoietic factors such as kit ligand, IL-11, VEGF, F1k2/F1t3 ligand, and the like. Differentiation into neuronal lineage may be induced using EGF and bFGF as described in published U.S. Patent Application No. 20050260747.

The hESC lines can be used in a transplant setting in the treatment (including prevention) of various conditions that affect one or more lineages. Examples of such conditions include but are not limited to Parkinson's disease (dopaminergic neurons), Alzheimer's disease (neural precursors), Huntington's disease (GABAergic neurons), blood disorders such as leukemia, lymphoma, myeloma and anemia (hematopoietic cells), side-effects of radiation e.g., in transplant patients (hematopoietic precursors), myocardial infarction, ischemic cardiac tissue or heart-failure (partially- or fully-differentiated cardiomyocytes), muscular dystrophy (skeletal muscle cells), liver cirrhosis or failure (hepatocytes), chronic hepatitis (hepatocytes), diabetes including type I diabetes (insulin-producing cells such as islet cells), ischemic brain damage (neurons), spinal cord injury (glial progenitor cells and motor neurons), amyotrophic lateral sclerosis (ALS) (motor neurons), orthopedic tissue injury (osteoblasts), kidney disease (kidney cells), corneal scarring (corneal stem cells), cartilage damage (chondrocytes), bone damage (osteogenic cells including osteocytes), osteoarthritis (chondrocytes), myelination disorders such as Pelizaeus-Merzbacher disease, multiple sclerosis, adenoleukodystrophies, neuritis and neuropathies (oligodendrocytes), and hair loss. References documenting the differentiation of embryonic stem cells into these various lineages include Bjorklund et al., 2002, PNAS USA 99:2344-2349 (dopaminergic neurons), West and Daley, 2004, Curr Opin Cell Biol 16:688-692; U.S. Pat. No. 6,534,052 B1; Kehat and Gepstein, 2003, Heart Fail Rev 8:229-236; Nir et al., 2003, Cardiovasc Res 58:313-323; U.S. Pat. Nos. 6,613,568 and 6,833,269. Transplant of differentiated cells and/or undifferentiated or partly differentiated embryonic stem cells is embraced by the invention.

The hESCs and/or their differentiated progeny may be introduced into a subject locally or systemically by any number of methods and routes. Local administration includes direct injection into particular sites in the body including organs and tissues whether normal or abnormal. Such local administration may be performed by direct needle injection. Systemic administration embraces parenteral (e.g., intravenous, intramuscular, subcutaneous, intraperitoneal, intra-tumor, intrathecal, etc.) and non-parenteral routes of administration.

The hESC lines and/or their differentiated progeny can be provided in pharmaceutical preparations. Such preparations are suited to in vivo administration, and are therefore minimally sterile and physiologically acceptable to the recipient. These preparations may generally comprise a pharmaceutically acceptable carrier. As used herein, a pharmaceutically acceptable carrier means a non-toxic material that does not interfere with the efficacy of the administered cells and/or other agents. Pharmaceutically acceptable carriers include diluents, fillers, salts, buffers, stabilizers, preservatives, solubilizers and other materials which are well-known in the art. Pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts. Pharmaceutical preparations may also contain other therapeutic agents. The invention also embraces pharmaceutical preparations which are formulated for local administration, such as implants. Exemplary bioerodible implants that are described in PCT International Application No. PCT/US/03307 (Publication No. WO 95/24929).

The invention further contemplates the ability to transduce embryonic stem cells and/or their differentiated progeny with particular nucleic acids, thereby giving rise to genetically modified stem cells and progeny. If intended for transplant, these cells can then be used for example to generate particular factors or to complement particular mutations in the recipient. Transduction of hESC lines is also taught in US Patent Application Publication No. 20050079616.

As used herein, “transduction of embryonic stem cells” refers to the process of transferring exogenous genetic material into an embryonic stem cell. The terms “transduction”, “transfection” and “transformation” are used interchangeably herein, and refer to the process of transferring exogenous genetic material into a cell. As used herein, “exogenous genetic material” refers to nucleic acids or oligonucleotides, eithernatural or synthetic, that are introduced into the cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization, proteins that confer intracellular localization and/or enhance intracellular half life, and the like. Polymeric delivery systems also have been used successfully to deliver nucleic acids into cells, as is known by those skilled in the art.

One method of introducing exogenous genetic material into cells is through the use of replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art. A preferred retroviral expression vector includes an exogenous promoter element to control transcription of the inserted exogenous gene. Such exogenous promoters include both constitutive and inducible promoters.

The major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into cells. The selection of an appropriate vector to deliver a therapeutic agent for a particular condition amenable to gene replacement therapy and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works with the coding sequence (in cis) to change the basal transcription level dictated by the promoter. Preferably, the exogenous genetic material is introduced into the genome immediately downstream from the promoter so that the promoter and coding sequence are operatively linked so as to permit transcription of the coding sequence.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector preferably includes a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

The selection and optimization of a particular expression vector for expressing a specific gene product in a cell is accomplished by obtaining the gene, preferably with one or more appropriate control regions (e.g., promoter, insertion sequence); preparing a vector construct comprising the vector into which is inserted the gene; transfecting or transducing cultured cells in vitro with the vector construct; and determining whether the gene product is present in the cells.

TABLE 1
Human Gene Therapy Protocols Approved by RAC: 1990-1994

| | | |
|---|---|---|
| Severe combined | Autologous lymphocytes transduced with human | Jul. 31, 1990 |
| immune deficiency | ADA gene | |
| (SCID) due to | | |
| ADA deficiency | | |
| Advanced cancer | Tumor-infiltrating lymphocytes transduced with tumor | Jul. 31, 1990 |
| | necrosis factor gene | |
| Advanced cancer | Immunization with autologous cancer cells transduced | Oct. 07, 1991 |
| | with tumor necrosis factor gene | |
| Advanced cancer | Immunization with autologous cancer cells transduced | Oct. 07, 1991 |
| | with interleukin-2 gene | |
| Asymptomatic patients | Murine Retro viral vector encoding HIV-1 genes | Jun. 07, 1993 |
| infected with HIV-1 | [HIV-IT(V)] | |
| AIDS | Effects of a transdominant form of rev gene on AIDS | Jun. 07, 1993 |
| | intervention | |
| Advanced cancer | Human multiple-drug resistance (MDR) gene transfer | Jun. 08, 1993 |
| HIV infection | Autologous lymphocytes transduced with catalytic | Sep. 10, 1993 |
| | ribozyme that cleaves HIV-1 RNA (Phase I study) | |
| Metastatic | Genetically engineered autologous tumor vaccines | Sep. 10, 1993 |
| melanoma | producing interleukin-2 | |
| HIV infection | Murine Retro viral vector encoding HIV-IT(V) genes | Dec. 03, 1993 |
| | (open label Phase I/II trial) | |
| HIV infection | Adoptive transfer of syngeneic cytotoxic T lymphocytes | Mar. 03, 1994 |
| (identical twins) | (Phase I/II pilot study) | |
| Breast cancer (chemo- | Use of modified Retro virus to introduce chemotherapy | Jun. 09, 1994 |
| protection during | resistance sequences into normal hematopoietic cells | |
| therapy) | (pilot study) | |
| Fanconi's anemia | Retro viral mediated gene transfer of the Fanconi anemia | Jun. 09, 1994 |
| | complementation group C gene to hematopoietic progenitors | |
| Metastatic | Autologous human granulocyte macrophage-colony | ORDA/NIH |
| prostate | stimulating factor gene transduced prostate cancer vaccine | Aug. 03, 1994* |
| carcinoma | | |
| Metastatic breast | In vivo infection with breast-targeted Retro viral vector | Sep. 12, 1994 |
| cancer | expressing antisense c-fos or antisense c-myc RNA | |
| Metastatic | Non-viral system (liposome-based) for delivering human | Sep. 12, 1994 |
| breast cancer | interleukin-2 gene into autologous tumor cells | |
| (refractory or | (pilot study) | |
| recurrent) | | |
| Mild Hunter | Retro viral-mediated transfer of the iduronate-2-sulfatase | Sep. 13, 1994 |
| syndrome | gene into lymphocytes | |
| Advanced | Use of recombinant adenovirus (Phase I study) | Sep. 13, 1994 |
| mesothelioma | | |

*(first protocol to be approved under the accelerated review process; ORDA = Office of Recombinate DNA Activities)

Table 1 represents only examples of genes that can be delivered according to the methods of the invention. Suitable promoters, enhancers, vectors, etc., for such genes are published in the literature associated with the foregoing trials. In general, useful genes replace or supplement function, including genes encoding missing enzymes such as adenosine deaminase (ADA) which has been used in clinical trials to treat ADA deficiency and cofactors such as insulin and coagulation factor VIII. Genes which affect regulation can also be administered, alone or in combination with a gene supplementing or replacing a specific function. For example, a gene encoding a protein which suppresses expression of a particular protein-encoding gene can be administered.

The invention still further contemplates screening various compounds for their effects on the hESCs provided herein. The compounds can be screened for their ability to maintain the hESC lines in an undifferentiated state, or to induce differentiation of the hESCs, or to otherwise modify the cells. Some screening aspects may be directed to testing the therapeutic efficacy of candidate compounds. Depending on the particular compounds being screened, the assay readouts will vary. For example, in some assays, the readout will be maintenance and/or increase in hESC numbers while in others the readout will be production of differentiated progeny (optionally with concomitant decrease in hESC numbers). Differentiation of hESCs can also be followed via changes in expression profiles. For example, differentiation of hESCs may be identified by down regulation of SSEA-3 and SSEA-4 expression and up regulation of SSEA-1 expression. Still other assays may include a cell viability or alternatively a cell death readout. Such assays may then provide in vitro readouts that potentially correlate with the toxicity and/or efficacy which the tested compounds would exhibit in human subjects. Thus, the effect of the agent on the hESC line or its differentiated progeny in vitro is a form of surrogate marker or readout for how the agent will function in vivo. The lines can be further used as a model system in which to develop a personalized therapeutic regimen for a patient who may be genetically or histocompatibly similar to the line.

The present invention is further illustrated by the following Examples, which in no way should be construed as further limiting.

### EXAMPLES

### Derivation of hESC Lines

The first two human embryonic stem cell lines (VAL-1 and VAL-2) have been derived in Spain with long-term cryopreserved embryos under animal-free conditions. All embryos frozen for >5 years were donated after informed consent for stem cell derivation, according to Spanish law 45/2003 November 21. Forty human embryos that had been cryopreserved at day 2 of development for >5 years were thawed. Six embryos did not survive this process: 5 degenerated, and the zona of 1 embryo fractured. A total of 15 of 34 embryos (44.1%) arrested during the initial stages of development: 12 stopped growing on the first day (35.3%) and 3 (8.8%) subsequently. Additionally, 3 pseudo-blastocysts formed (8.8%). In all, 16 blastocysts were obtained (47.1%) and classified according to Gardner et al. Fertil Steril 1998 69:84-88. Of those, 11 blastocysts (68.7%) had an inner cell mass (ICM) grade of either A or B.

A complete description of the production of placental fibroblast lines has been described by Genbacev et al. Fertil Steril 2005 83:1517-1529. Human placental fibroblasts obtained from early-gestation human placentas support propagation of established hESC lines. In all respects, placental fibroblasts were comparable to mouse fibroblasts as hESC feeders. Subsequently, one qualified placental fibroblast line was used as a feeder layer for the derivation of new hESC.

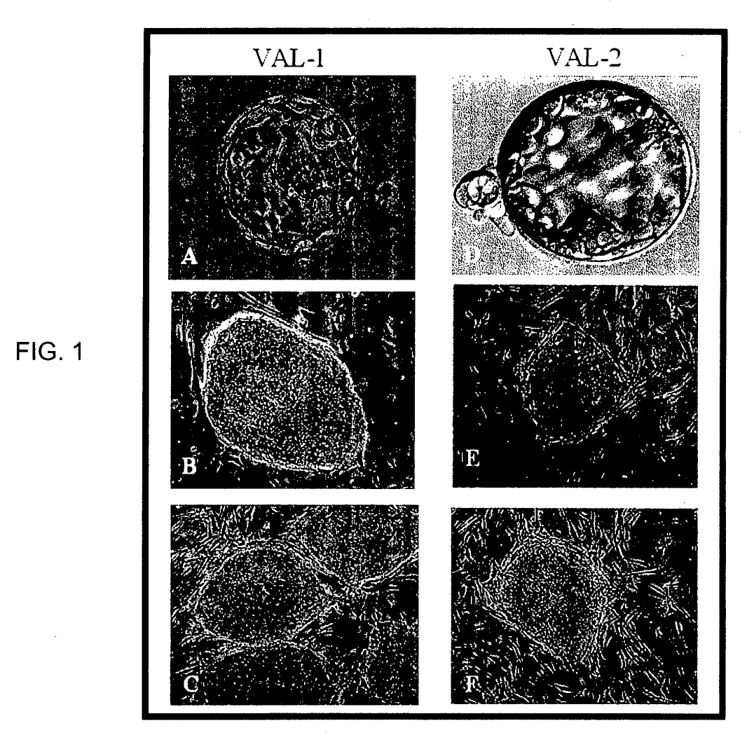

Briefly, the zona pellucida was removed with acid Tyrode's solution as described in Genbacev et al., and the blastocysts (see FIGS. 1A and D) were plated on feeders formed from irradiated human placental fibroblasts in defined medium: 79% knockout Dulbecco's minimum essential medium (DMEM) (Gibco/BRL, Paisley, United Kingdom), 20% knockout recombinant serum (RS) (Gibco/BRL), 1 mmol/L glutamine (Gibco/BRL), 0.1 mmol/L β-mercaptoethanol (Sigma, St. Louis, Mo.), 1% nonessential amino acids stock (Gibco/BRL), containing 12 ng/mL human basic fibroblast growth factor (Invitrogen; Life Technologies, Carlsbad, Calif.). The derivation process was carried out in accordance with GMP.

Within the first 2 days, 14 blastocysts attached (87.5%) to the human feeders, and in two cases outgrowths with hESC-like morphology appeared 5-7 days after plating. Eighteen and 21 days later, individual expanded ICMs (see FIGS. 1B and E) were mechanically dissected into three pieces and allowed to reattach in the same well. After 6-8 days, the colonies were once again mechanically divided and then transferred to fresh human placental fibroblasts feeders. Successful derivation was associated with the appearance of round cells with prominent nucleoli (see FIGS. 1C and F), whereas cells with a stromal-like morphology either died or disappeared after passaging. Thus far, the new hESC lines have been cultured and mechanically dissected to the 120th (VAL-1) and 85th (VAL-2) passage under the same conditions that were used for the derivation (i.e., on human placental fibroblast feeders in defined medium without serum). Additionally, VAL-1 and VAL-2 have been successfully cryopreserved and thawed using the conventional slow freezing method with 10% dimethyl sulfoxide as a cryoprotectant as described by Bongso et al. Hum. Reprod. 11:2110-2117, 1994.

These data suggest a derivation efficiency of 5% per frozen embryo or 12.5% per blastocyst.

### Characterization of hESC Lines

After passaging, VAL-1 and VAL-2 colonies had a larger surface area, appeared thinner and flatter, and had straight defined boundaries, giving to the colonies either angular or circular edges (see FIGS. 1C and F). Under high magnification, individual hESC on human feeders were small and round, with prominent nucleoli, a typical feature of these cells.

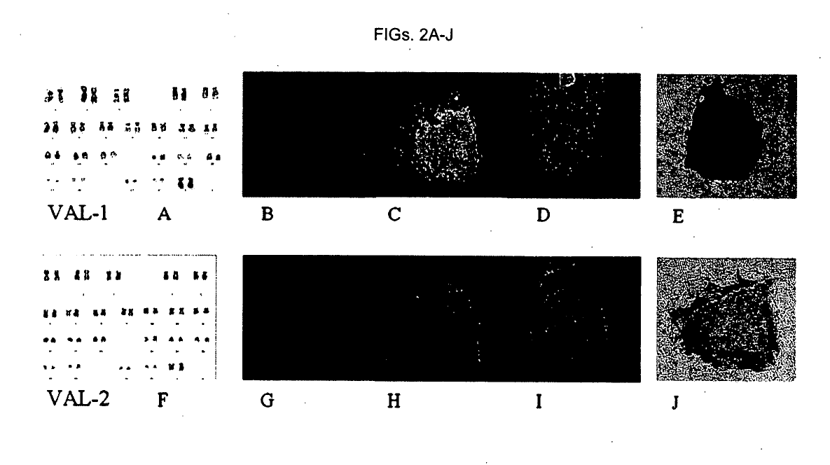

Starting at passage 4, the cells were karyotyped each time the colonies were divided. hESC were incubated in hES medium, supplemented with 0.2 μg/ml colcemid (ROCHE, 10 μg/ml stock solution) at 37° C. for 30 minutes, and subsequently washed three times with 2 ml PBS+Ca+Mg. A minimum of 20 colonies were mechanically dissected on PBS from the feeder layer, collected in 2 ml of 1×trypsin-EDTA and incubated at 37° C. for 5 minutes. The final mixture of cells was pipetted several times at the end of the incubation, in order to ensure the total disintegration in single cells. The trypsin activity was stopped with 4 ml of hES medium and spun at 1800 rpm for 10 minutes. After that, the supernatant was discarded and the pellet was carefully resuspended and incubated in 1 ml of pre-warmed potassium chloride solution (KCl, 0.075 M) for 10 minutes at 37° C. The cells were pre-fixed with 1 ml of Carnoy fixative solution ( 3 vols. methanol: 1 vol. Acetic acid) at −20° C., and immediately spun at 1800 rpm for 10 minutes. Finally, the supernatant was discarded and resuspended again in Carnoy solution. Cytogenetic analyses of at least 20 metaphase spreads and five banded karyotypes were evaluated for chromosomal rearrangements using the GTG-banding method by two qualified geneticists at Prenatal Genetics (Barcelona, Spain). Every analysis showed that both cell lines maintained a normal 46, XX karyotype (see FIGS. 2A and F).

Fixed hESC colonies were exposed to primary antibodies specific for Tumor Rejection Antigens TRA-1-60 and TRA-1-81 (generously provided by Peter Andrews, Sheffield University), and Stage-Specific Embryonic Antigen 4 (SSEA-4), (Chemicon, Temecula, Calif.), Alkaline phosphatase activity was demonstrated using a Vector Blue/Red substrate kit (Vector Laboratories, Burlingame, Calif.). Immunolocalization studies showed that VAL-1 and VAL-2 expressed SSEA-4 (Chemicon; Temecula, Calif.) (see FIGS. 2B and G), TRA-1-60 (see FIGS. 2C and H) and TRA-1-81 (see FIGS. 2D and I). Alkaline phosphatase activity was also detected (see FIGS. 2E and J).

Real-time PCR of various markers was also performed. Total RNA from VAL-1 and VAL-2 grade A colonies was extracted using TRIzol Reagent (Invitrogen) according to the manufacturer's instructions for small-scale quantities of cells, and RNA concentration was assessed using a spectrophotometer (BioRad). Total RNA (1 μg) from each sample was used for first-strand cDNA synthesis by using the Advantage RT-for-PCR kit (BD Biosciences) following the manufacturer's protocol. PCR primers were designed using freely available web-based software (Primer3, Genefisher). PCR reactions using 1 μg total cDNA as template were carried out as follows: denaturation a 94° C. for 4 minutes, and cycled 40 times at 94° C. for 1 minute, 55° C. for 1 minute and 72 ° C. for 1 minute. A final extension at 72° C. for 10 minutes was performed after cycling. PCR products were resolved on 2% agarose gels, stained with ethidium bromide, and visualized in a transilluminator (BioRad). These RT-PCR studies demonstrated that both cell lines were positive for Oct-4, Rex-1, Cripto, Thy-1, and Nanog and were negative for classical differentiation markers: Matn1, Amylase, and Dbh.

Telomerase activity of VAL-1 and VAL-2 was analyzed using a TRAP_{EZE}® Telomerase Detection Kit (Chemicon) followed by staining with SYBR® (Molecular Probes, Eugene, Ore.). Briefly, colonies (50-100) were harvested, washed once in Ca++- and Mg++-free PBS and immediately resuspended in lysis buffer. After treatment in ice and centrifugation, samples were subjected to a PCR reaction following the manufacturer's instructions. PCR products were run in a polyacrylamide gel (15% TBE, BioRad) in non-denaturing conditions, and amplified fragments were then stained in SYBR green for visualization in a transilluminator. The negative controls were obtained by heat inactivation of the lines.

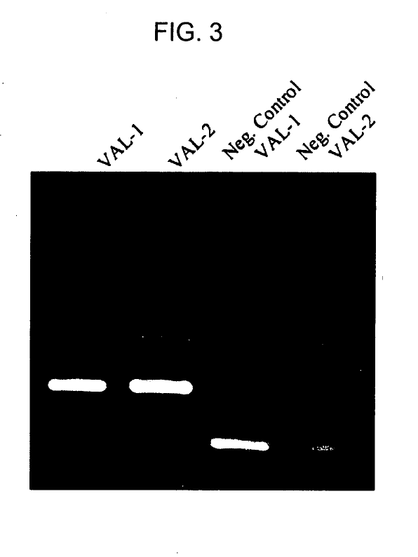

The end-product contained a ladder of amplification products with 6 bp increments starting at 50 bps (see FIG. 3). The 36 bp band corresponds to the internal polymerase chain reaction control, and the 50 bp band to telomerase activity which increases in 6 bp bands in immortal cells.

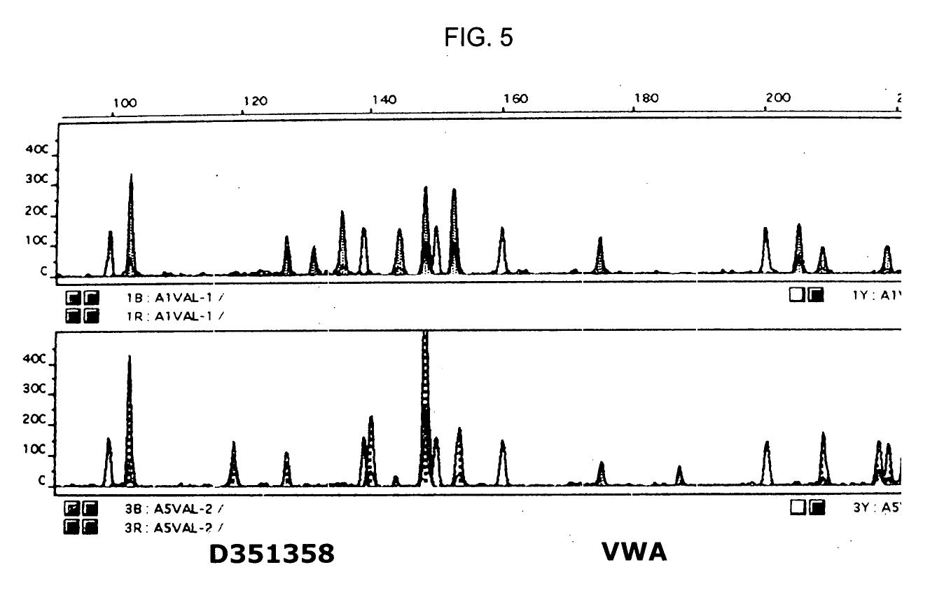

Further analysis of VAL-1 and VAL-2 revealed that the lines were immunologically identical for the HLA A2, A23, B44, B40, CW4, CW5, DR7, DR15, DQ2 and DQ6 markers. This level of histocompatibility indicates that the lines can be used jointly in a therapeutic regimen with little or no risk of immunological rejection. The lines however are not genetically identical, as shown in FIGS. 4, 5 and 6.

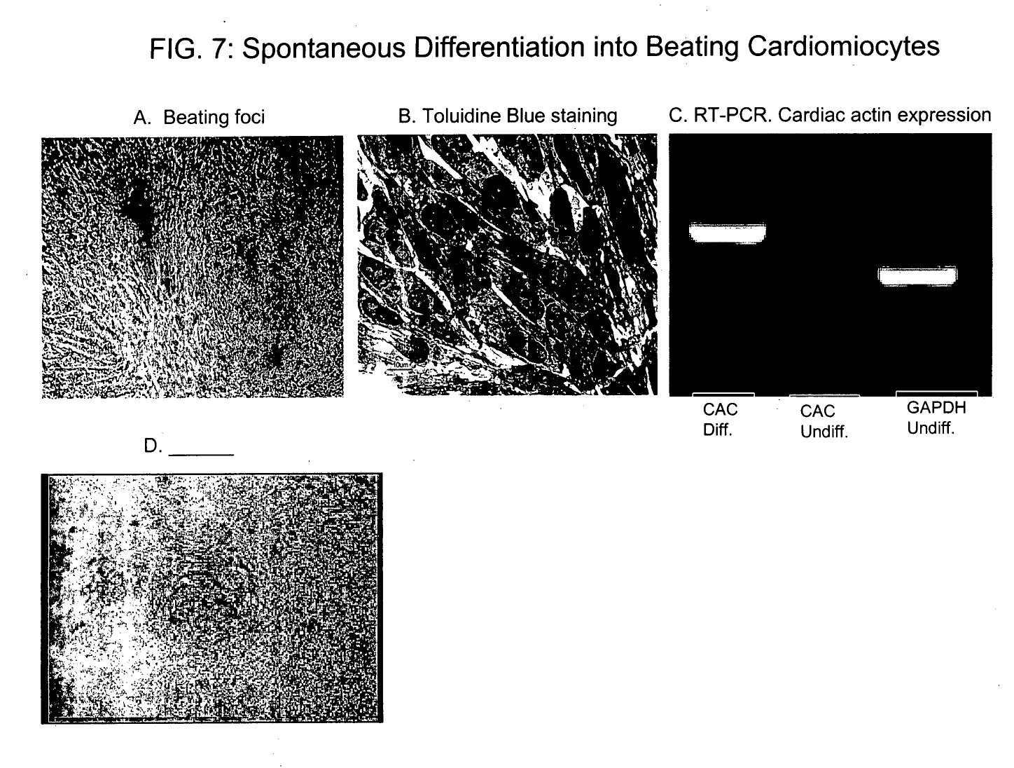

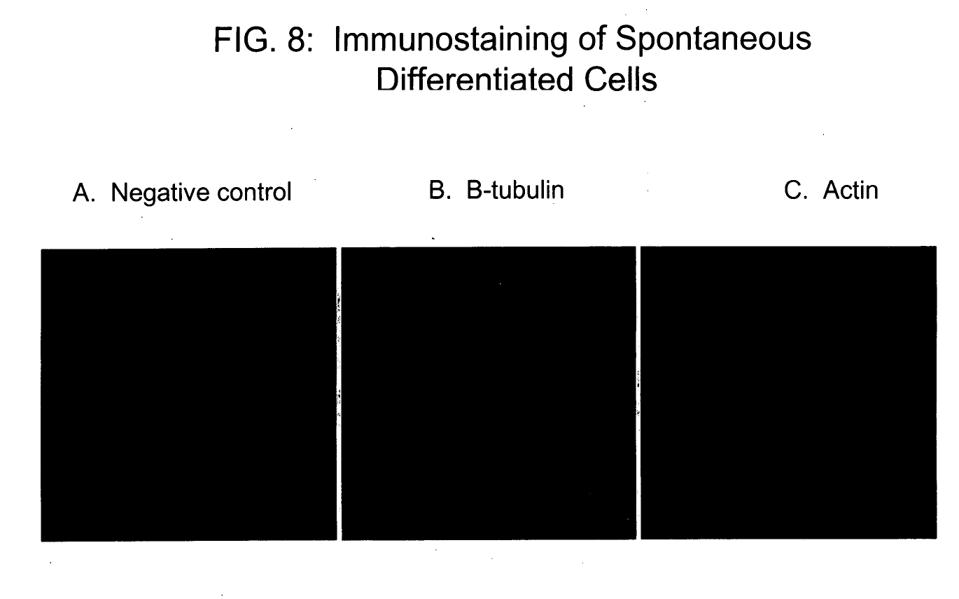

VAL-1 and VAL-2 were also evaluated for their spontaneous differentiation profiles. Colonies were dissociated by collagenase IV treatment for 5 minutes at 37° C. and then cultured in suspension on low-attachment 6-well culture plates. The differentiation medium consisted of 80% DMEM, 20% FBS (Hyclone), 1 mM L-glutamine, 0.1 mM β-mercaptoethanol, and 1 mM MEM nonessential amino acids. After 4 days in suspension, the embryoid bodies were transferred onto poly-L-omithine-coated chamber slides and cultured for an additional 10-14 days. The cultures were fixed with 4% paraformaldehyde for 20 minutes before immunolocalization analyses. To assess the expression of markers associated with differentiation of the three germ layers, embryoid bodies were incubated with for example mouse (ascites) anti-α-fetoprotein (diluted 1:500; Sigma), mouse (ascites) anti-p-tubulin III (diluted 1:1,000; Sigma), or mouse anti-smooth muscle actin (10.7 μg/mL; Dako). Negative controls included omission of the primary antibodies and incubation with non-specific IgG. VAL-1 showed the ability to differentiate spontaneously into myocardiocyte-like cells (FIGS. 7A-D), and both VAL-1 and VAL-2 showed the ability to differentiate spontaneously into neuron-like cells (FIG. 8B), and muscle-like cells (FIG. 8C).

The gene expression profiles of spontaneously differentiated progeny of the lines were also analyzed. As shown in Table 2, the lines are able to express spontaneously genes associated with endoderm, mesoderm and ectoderm lineages.

TABLE 2
Spontaneous Differentiation Gene Expression Profile

| Gene | Germ Layer | VAL-1 | VAL-2 |
|---|---|---|---|
| neurofilament heavy chain | Ectoderm | − | − |
| keratin | Ectoderm | ? | + |
| dopamine β hydroxylase | Ectoderm | − | − |
| enolase | Mesoderm | + | − |
| cartilage matrix protein | Mesoderm | ? | − |
| renin | Mesoderm | − | − |
| kallikrein | Mesoderm | + | + |
| wilms tumor 1 | Mesoderm | + | + |
| cardiac actin | Mesoderm | + | + |
| δ-globin | Mesoderm | + | + |
| β-globin | Mesoderm | + | + |
| albumin | Endoderm | − | − |
| α1 anti-trypsin | Endoderm | + | − |
| amylase | Endoderm | + | + |
| PDX-1 | Endoderm | + | [symbol] |
| insulin | Endoderm | − | − |
| α-feto protein | Endoderm | + | ? |
| GAPDH | Housekeeping | + | + |

“ [symbol] ” indicates inconclusive results

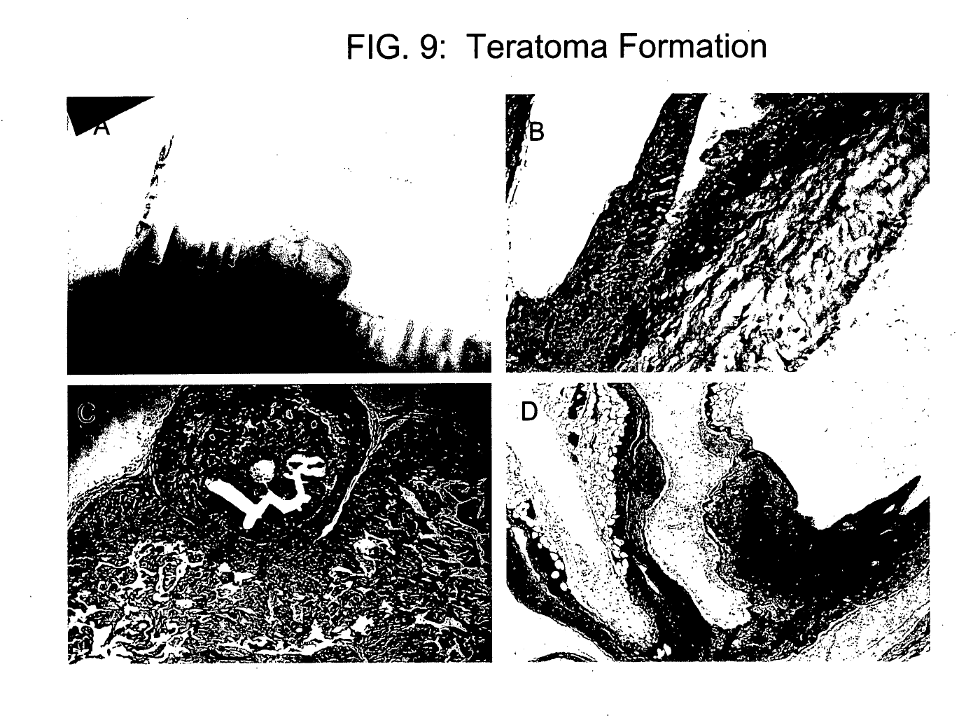

The ability of the lines to form teratomas in vivo was also evaluated. 2×10⁵ undifferentiated hESCs were administered by intratesticular injection into CB 17 severe combined immunodeficient (SCID) mice (n=22). Tissues were harvested after 11 weeks, and histologic analysis was performed. Both lines formed teratomas in recipient mice. An example of a resultant teratoma and its histological analysis are shown in FIGS. 9A-D and 10A-L. These analyses demonstrate the ability of the lines to differentiate into one or more endoderm (e.g., intestinal epithelium, etc.), one or more ectoderm (e.g., neuronal rosettes, etc) and one or more mesoderm (e.g., cartilage, etc.) lineages.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. All references, patents and patent applications that are recited in this application are incorporated by reference herein in their entirety.

What is claimed is:

1. An isolated human embryonic stem cell population wherein said cell population (1) has a normal karyotype after at least 85 passages; (2) expresses Stage Specific Embryonic Antigen (SSEA) 4, Tumor Rejection Antigen (TRA)-1-60 and TRA-1-81; (3) is alkaline phosphatase positive; (4) expresses Oct-4, Rex-1, Cripto, Thy-1 and Nanog; (5) is negative for Matn1, Amylase and Dbh; and (6) has telomerase activity.

2. An isolated human embryonic stem cell population wherein said cell population is VAL-1, and progeny thereof.

3. An isolated human embryonic stem cell population wherein said cell population is VAL-2, and progeny thereof.

4. An isolated human embryonic stem cell population wherein said cell population has the characteristics of the population of claim 2.

5. An isolated human embryonic stem cell population wherein said cell population has the characteristics of the population of claim 3.

6. (canceled)

7. The isolated human embryonic stem cell population of claim 1, wherein the population is passaged at least 50 times.

8.-11. (canceled)

12. An isolated human embryonic stem cell population wherein the population is a genetically modified version of the VAL-1 human embryonic stem cell line.

13. An isolated human embryonic stem cell population wherein the population is a genetically modified version of the VAL-2 human embryonic stem cell line.

14. A culture comprising the human embryonic stem cell population of claim 1.

15. The culture of claim 14, further comprising human feeder cells.

16. The culture of claim 15, wherein the human feeder cells are human placental feeder cells.

17. The culture of claim 14, wherein the culture is a serum-free culture.

18. The culture of claim 14, further comprising fibroblast growth factor (FGF).

19. The culture of claim 18, wherein the FGF is basic FGF.

20. The culture of claim 14, wherein the culture is feeder-free.

21. The culture of claim 15, wherein the human placental feeder cells are mitotically inactivated by irradiation.

22. A method of culturing a human embryonic stem cell population comprising
culturing the human embryonic stem cell population of claim 1 in the presence of human feeder cells in a serum-free medium.

23.-27. (canceled)

28. A method of differentiating a human embyronic stem cell population in vitro comprising
exposing the human embryonic stem cell population of claim 1 to differentiation conditions for a time sufficient to allow differentiation of the embryonic stem cell population into differentiated cells.

29.-32. (canceled)

33. A method of differentiating a human embryonic stem cell population comprising
introducing the human embryonic stem cell population of claim 1 into a human subject.

34.-42. (canceled)

43. A composition comprising a human embryonic stem cell from the human embyronic stem cell population of claim 1.
